(19) United States
(12) Patent Application Publication
(10) Pub. No.: US 2014/0200265 A1
(43) Pub. Date: Jul. 17, 2014

(54) USAGE OF 1,3,5,7-TETRAHYDROXY-8-ISOPRENYLXANTHONE FOR TREATING INFLAMMATION

(71) Applicant: Hong Kong Baptist University, Hong Kong (HK)

(72) Inventors: Hongxi Xu, Hong Kong (HK); Dandan Zhang, Hong Kong (HK); Hong Zhang, Hong Kong (HK); Kaixian Chen, Hong Kong (HK); Zhaoxiang Bian, Hong Kong (HK); Chengyuan Lin, Hong Kong (HK); Dajian Yang, Hong Kong (HK); Shilin Chen, Hong Kong (HK); Aiping Lu, Hong Kong (HK); Albert Sun Chi Chan, Hong Kong (HK)

(73) Assignee: Hong Kong Baptist University, Hong Kong (HK)

(21) Appl. No.: 14/150,726

(22) Filed: Jan. 8, 2014

Related U.S. Application Data
(60) Provisional application No. 61/751,308, filed on Jan. 11, 2013.

(51) Int. Cl. C07D 311/86

(52) U.S. Cl. CPC C07D 311/86
USPC 514/455; 549/392

(57) ABSTRACT

This invention relates to the use of a natural compound from natural sources for its therapeutic uses. More particularly, it relates to a compound 1,3,5,7-tetrahydroxy-8-isoprenylxanthone (TI, FIG. 1), that is naturally occurring in the plant of Garcinia esculenta Y. H. Li, and its biological activity of inhibiting nitric oxide production and microRNA 155 expression in stimulated macrophages, which can be developed as potential anti-inflammatory drugs.

### CROSS-REFERENCE TO RELATED APPLICATIONS

This application claims the benefit of U.S. Provisional Patent Application No. 61/751,308 filed Jan. 11, 2013; the disclosure of which is incorporated herein by reference in its entirety.

### FIELD OF INVENTION

This invention relates to a chemical entity isolated from natural sources for its therapeutic uses. More particularly, it relates to a compound 1,3,5,7-tetrahydroxy-8-isoprenylxanthone (TI, FIG. 1), that is naturally occurring in the plant of Garcinia esculenta Y. H. Li, and its biological activity of inhibiting nitric oxide production and microRNA 155 expression in stimulated macrophages, which can be developed as potential anti-inflammatory drugs.

### BACKGROUND OF INVENTION

The normal inflammatory process represent a protective mechanism of the body initially, excessive and chronic inflammation results in damage of the cells and tissues. The merging evidences support the hypotheses that inflammation plays a key role in various chronic pathological conditions including atherosclerosis, stroke, cancer and neurodegenerative diseases. The massive amounts of nitric oxide (NO) produced in response to bacterial LPS or cytokines play an important role in the inflammatory conditions. Natural products have played a significant role in anti-inflammatory drug discovery and development. Drug designs that decrease NO production have a therapeutic effect in the treatment of septic shock, as well as many other inflammatory and immune disorders.

Nitric oxide (NO) chemically belongs to free radical, which is synthesized from L-arginine by nitric oxide synthase (NOS). Constitutive isoform of NOS (cNOS) includes eNOS and nNOS, which generate nanomolar concentration of NO and mediate various physiological functions. However, the inducible isoform of the NOS (iNOS) in the majority of cases only expresses upon the stimulation by inflammatory factors, and can produce excess amount of NO, which promotes inflammatory reactions and may have pathological consequences. It is notable that various inflammatory stimuli can activate distinct signaling pathways that converge to trigger the expression of iNOS.

MicroRNAs (miRNAs) are non-coding RNAs of 22 nucleotides that mediate posttranscriptional gene repression. A wide variety of miRNAs are involved in a multitude of cellular and developmental processes. The LPS-stimulated RAW264.7 cells and LPS-injected mice had an increase of miR-155 and a decrease of miR-125b associated with a higher level of TNF-α. These data suggest that miR-155 regulates inflammatory mediators in innate immune responses and regulating inflammatory mediators.

Zhang Z et al in Zhang Z, ElSohly H N, Jacob M R, et al. showed that natural products inhibiting Candida albicans secreted aspartic proteases from Tovomita krukovii. Planta Med. 2002 January; 68 (1):49-54 indicated this compound was received from Tovomita krukovii and showed inhibitory effects against Candida albicans secreted aspartic proteases (SAP) with IC50 values of 25 microg/ml. But none evaluated that compound on inflammatory effect.

An objective of this invention is to use 1,3,5,7-tetrahydroxy-8-isoprenylxanthone as a medicament to treat inflammation related diseases. Our results indicated that it could decrease the over-production of nitric oxide in cell inflammation model, LPS and IFN-γ stimulated macrophages. The mechanisms underlying the anti-inflammatory effects of 1,3,5,7-tetrahydroxy-8-isoprenylxanthone include the inhibition of microRNA 155, which is a high expression in stimulated macrophages.

Citation or identification of any reference in this section or any other section of this application shall not be construed as an admission that such reference is available as prior art for the present application.

### SUMMARY OF INVENTION

Accordingly, an objective of this invention is to provide a natural compound from a natural source that exhibits potent anti-inflammatory effect, and thus, have potential to be developed as anti-inflammatory drugs.

In accordance with one aspect of the present invention, there is provided a composition for treating inflammation comprising an effective amount of a compound with the chemical structure of

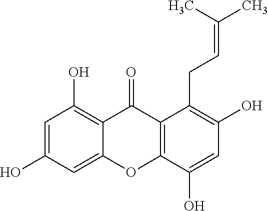 [SMILES: CC(C)=CCc1c(O)cc(O)c2oc3cc(O)cc(O)c3c(=O)c12]

In accordance with another aspect of the present invention, there is provided a compound 1,3,5,7-tetrahydroxy-8-isoprenylxanthone, that is naturally occurring in the plant of Garcinia esculenta Y. H. Li, and its biological activity of inhibiting nitric oxide production, and thus, can be used as active compounds in drugs for anti-inflammatory treatment.

In accordance with yet another aspect of the present invention, there is provided a compound 1,3,5,7-tetrahydroxy-8-isoprenylxanthone, that is naturally occurring in the plant of Garcinia esculenta Y. H. Li, and its biological activity of inhibiting microRNA 155 expression in stimulated macrophages, and thus, can be used as active compounds in drugs for anti-inflammatory treatment.

Those skilled in the art will appreciate that the invention described herein is susceptible to variations and modifications other than those specifically described.

The invention includes all such variation and modifications. The invention also includes all of the steps and features referred to or indicated in the specification, individually or collectively and any and all combinations or any two or more of the steps or features.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

Other aspects and advantages of the invention will be apparent to those skilled in the art from a review of the ensuing description.

### BRIEF DESCRIPTION OF DRAWINGS

The above and other objects and features of the present invention will become apparent from the following description of the invention, when taken in conjunction with the accompanying drawings, in which:

FIG. 1 shows the structure of 1,3,5,7-tetrahydroxy-8-isoprenylxanthone

FIG. 2 shows effect of TI on expression of miRNA-155 in stimulated RAW264.7 cells.

### DETAILED DESCRIPTION OF INVENTION

The present invention is not to be limited in scope by any of the specific embodiments described herein. The following embodiments are presented for exemplification only.

Reagents

Murine recombinant IFN-γ was purchased from EMD Millpore Corporation (Temecula, Calif., U.S.A.). Lipopolysaccharide (LPS; Escherichia coli O111:B4), dimethyl sulfoxide (DMSO), sulfanilamine, N-(1-naphthyl)-enthylendiamine dihydrochloride, 3-(4,5-dimethylthiazol-2-yl)-2,5-diphenyltetrazoleum (MTT), and Indometacin were purchased from Sigma-Aldrich Co. LLC. RPMI-1640 was purchased from Gibco, Life Technologies Corporation (Grand Island, N.Y., U.S.A.). Fetal bovine serum (FBS) was purchased from Hyclone, Thermo Fisher Scientific Inc. (Victoria, Australia). TaqMan miRNA assays were purchased from ABI. All other chemicals were of analytical grade.

Cell Culture

RAW 264.7 cells were obtained from the American Tissue Culture Collection (ATCC). The cells were maintained in complete RPMI 1640 medium supplemented with 10% FBS at 37° C. in a humidified 5% CO2 atmosphere.

Assay for NO Accumulation in Stimulated Macrophages

RAW264.7 were maintained in RPMI 1640 medium supplemented with 10% FBS at 37° C. in a humidified 5% CO₂ atmosphere. Cells were plated in a 96-well plate (5×10³ cells/well) and allowed to attach overnight. The cells were then stimulated with 10 U/ml of IFN-γ and 100 ng/ml of LPS for 24 hours in the presence or absence of tested compound with final concentration of 50, 25, 12.5, 6.25, 3.125 μg/mL, and Indometacin as positive drug.

iNOS function was analyzed with Griess reaction and was applied for measurement of nitrite concentration in the culture supernatant. Briefly, 100 μl/well of sample was incubated with equal volume of Griess solution at room temperature for 10 min. The absorbance was evaluated at 540 nm using a calibration curve with a sodium nitrite standard. Percent inhibition was expressed as [1-(NO level of sample/NO level of vehicle treated-control)]×100 (Table 1). Table 1 shows the effect of TI on NO production in stimulated RAW264.7 cells.

Assay for microRNA155 Expression in Stimulated Macrophages

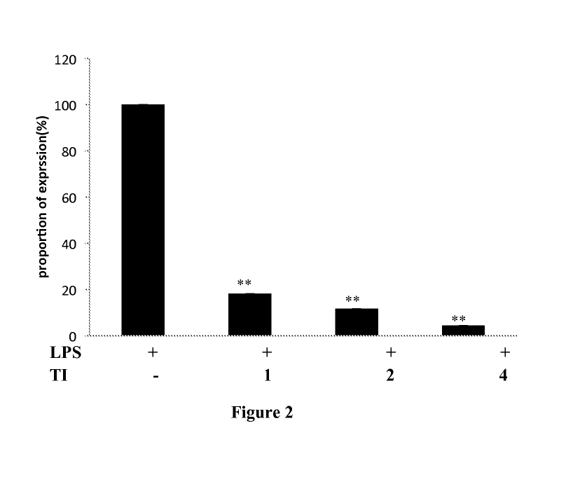

RAW264.7 cells were pretreated with tested sample (1,2,4 μg/mL,) for 1 hour and then stimulated for 24 hours. The cells were harvested. Total RNA was extracted and RNA concentrations were determined. Quantitative RT-PCR analyses on miRNAs were performed using TaqMan miR NA assays (Applied Biosystems, USA). 50 ng of RNA per reaction was subjected for RT reaction. RT reaction was carried out using the following conditions: 16° C. for 30 minutes, 42° C. for 30 minutes, and 85° C. for 5 minutes. Expression levels of the miRNAs were determined by qRT-PCR using TaqMan Expression Assays (Applied Biosystems, USA). RNU6 endogenous control was used for normalization, and expression levels were presented as ΔCt with standard deviation (FIG. 2).

Statistical Analysis

Results are presented as means±SD of three independent experiments. Student's test was used for each significant effect of treatment. Comparisons between multiple groups were performed with one-way ANOVA test. The threshold of significance was set at P<0.05 and P<0.01.

Results

Concentration-Dependent Inhibition of iNOS-NO Production by 1,3,5,7-tetrahydroxy-8-isoprenylxanthone

To further characterize the effect of 1,3,5,7-tetrahydroxy-8-isoprenylxanthone on iNOS-induced NO generation in RAW 264.7 cells, LPS/IFN-γ double stimulation model was adopted in observing the nitrite accumulation. A 24 hours inflammatory factors treatment triggered folds increase of nitrite concentration, as shown in Table 1. Application of 1,3,5,7-tetrahydroxy-8-isoprenylxanthone dose-dependently attenuated NO production at IC50 of 5.06 μM.

TABLE 1
The inhibitory effect on NO accumulation of sample

| Samples | IC₅₀(μg/mL) | IC₅₀(μM) |
|---|---|---|
| 1,3,5,7-tetrahydroxy-8-isoprenylxanthone | 1.66 | 5.06 |

Note:
The inhibitory effect on NO accumulation of Indometacin (1 μg/mL) is 46.51%.

Effect of 1,3,5,7-tetrahydroxy-8-isoprenylxanthone on Expression of Stimulating miR-155 Expression

As miR-155 plays an important role in the innate immune response or inflammation, the effect of 1,3,5,7-tetrahydroxy-8-isoprenylxanthone on the expression of miR-155 by qRT-PCR was evaluated. In FIG. 2, the cells were stimulated with 100 ng/ml of LPS and 10 U/ml of IFN-γ only or LPS plus different concentrations (1,2,4 μg/mL) of sample for 24 hours. Total RNA was isolated and the expression of miRNA-155 was determined by qRT-PCR. As shown in FIG. 2, 1,3,5,7-tetrahydroxy-8-isoprenylxanthone reduced LPS plus IFN-γ-induced miR-155 expression in a dose-dependent manner. RNU6B was used here as an endogenous control. The data represent the mean±SD of triplicate experiments. **p<0.01 compared with the LPS plus IFN-γ.

### CONCLUSION

The results indicated that it could decrease the over-production of nitric oxide in cell inflammation model, LPS and IFN-γ stimulated macrophages. The mechanisms underlying the anti-inflammatory effects of include 1,3,5,7-tetrahydroxy-8-isoprenylxanthone could the inhibition of microRNA 155, which is a high expression in stimulated macrophages.

### INDUSTRIAL APPLICATION

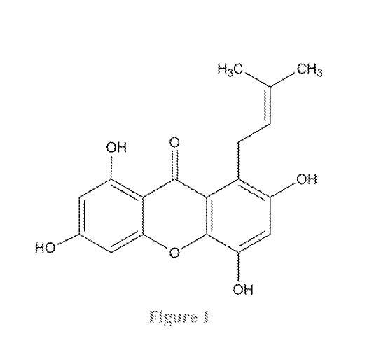

The present invention discloses a chemical entity isolated from natural sources for its therapeutic uses. More particularly, it relates to a compound 1,3,5,7-tetrahydroxy-8-isoprenylxanthone (TI, FIG. 1), that is naturally occurring in the plant of Garcinia esculenta Y. H. Li, and its biological activity of inhibiting nitric oxide production and microRNA 155 expression in stimulated macrophages, which can be developed as potential anti-inflammatory drugs

If desired, the different functions discussed herein may be performed in a different order and/or concurrently with each other. Furthermore, if desired, one or more of the above-described functions may be optional or may be combined.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the reminder of the disclosure in any way whatsoever. Without further elaboration, it is believed that one skilled in the art can, based on the description herein, utilize the present invention to its fullest extend. All publications recited herein are hereby incorporated by reference in their entirety.

What we claim:

1. A composition for treating inflammation, comprising a compound with the chemical structure of

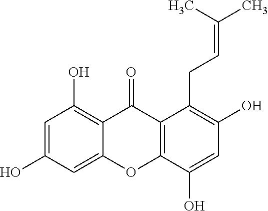 [SMILES: CC(C)=CCc1c(O)cc(O)c2oc3cc(O)cc(O)c3c(=O)c12]

2. The composition according to claim 1, wherein said compound comprising 1,3,5,7-tetrahydroxy-8-isoprenylxanthone.

3. The composition according to claim 1, wherein said compound is extracted from a natural plant.

4. The composition according to claim 3, wherein the natural plant comprising Garcinia species.

5. The composition according to claim 4, wherein said Garcinia species comprising Garcinia esculenta Y. H. Li.

6. The composition according to claim 1, wherein the treatment comprising the inhibition of nitric oxide production, microRNA 155, or combination thereof expression in stimulated macrophages.

7. A use of the composition according to claim 1 for manufacture of a medicament for treatment of inflammation.

8. A method of treating inflammation using composition according to claim 1 by administering said composition to a subject in need of such treatment.
